(19) United States
(12) Patent Application Publication
(10) Pub. No.: US 2005/0100597 A1
(43) Pub. Date: May 12, 2005

(54) Diclofenac-based composition for the topical treatment of oropharyngeal cavity disorders

(76) Inventor: Mario Pinza, Corsico (Milano) (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/505,178

(22) PCT Filed: Apr. 16, 2003
(86) PCT No.: PCT/EP03/04044

(30) Foreign Application Priority Data
May 10, 2002 (IT) MI2002A000986

(51) Int. Cl. A61K 31/205; A61K 31/195; A61K 9/20

(52) U.S. Cl. 424/464; 514/554; 514/567

(57) ABSTRACT

A composition for the topical treatment of oropharyngeal cavity disorders, comprising an aqueous solution of the salt of diclofenac with trimethamine, in which the amount of the said salt is of from 0.1% to 0.2% (w/w) and the pH is adjusted between 7 and 8.

The present invention relates to a diclofenac-based composition for the topical treatment of oropharyngeal cavity disorders.

It is known that diclofenac [2-(2,6-dichloroanilino)phenylacetic acid] is a widely-used pharmaceutical product with anti-inflammatory, antipyretic and analgesic properties. It is mainly administered systemically in unmodified form or in the form of a salt thereof with mineral or organic bases.

However, its salts are virtually insoluble in water.

Example 2 of U.S. Pat. No. 4,407,824 describes the preparation of the salt of diclofenac with tromethamine [tris(hydroxymethyl)methylamine], but does not specify its solubility in water and does not give an example of any pharmaceutical form containing the abovementioned salt.

The problem of the insolubility in water of diclofenac salts is also acknowledged in EP-A-0 521 393, which proposes to solve the said problem by means of the choline salt. This salt is described as a compound that is surprisingly soluble in water and suitable, inter alia, also for the preparation of mouthwashes.

However, the choline salt has the typical drawbacks of choline, which is well known for its unpleasant odour and taste.

These drawbacks are particularly unfavourable in the case of compositions for the topical treatment of oropharyngeal cavity disorders, for instance mouthwashes and oral sprays, which need to remain in contact with the mucosae for a relatively long period of time in order to exert their therapeutic effect.

Despite the addition of large amounts of ingredients capable of masking its taste [0.5% (w/w) of acesulfame and 35% (w/w) of sorbitol], compositions for the topical treatment of oropharyngeal cavity disorders based on the salt of diclofenac with choline are relatively unpalatable.

There is therefore still a great need for a diclofenac-based composition of pleasant or at the very least neutral taste, for the topical treatment of oropharyngeal cavity disorders.

Although A. Fini et al. have reported that the solubility in water of the tromethamine salt is considered to be 0.167 g in 100 ml (European J. Pharm. Sci. 4, 231, 1996), the tests conducted by the present inventor have demonstrated that amounts of diclofenac ranging from 0.071 to 0.142 g do not dissolve in 100 ml of water even in the presence of stoichiometric amounts (from 0.029 to 0.058 g, respectively) of tromethamine (Comparative Examples 1 and 2).

Surprisingly, it has now been found that the above-mentioned compositions containing from 0.071 to 0.142 g of diclofenac with stoichiometric amounts (from 0.029 to 0.058 g, respectively) of tromethamine in 100 ml of water become clear and remain so for a long time if their pH is brought to 7-8 (Examples 1 and 2).

Also surprisingly, it has been found that the palatability of these solutions is good and that it is also very easy to improve it by means of modest amounts of standard flavouring agents and sweeteners.

One subject of the present invention is thus a composition for the topical treatment of oropharyngeal cavity disorders, characterized in that it comprises an aqueous solution of the salt of diclofenac with tromethamine, in which the amount of the said salt is of from 0.1% to 0.2% (w/w) and the pH is adjusted between 7 and 8.

The preferred concentration of the salt of diclofenac with tromethamine in the composition of the present invention is 0.1% (w/w).

Advantageously, the abovementioned mouthwash comprises other standard ingredients, for instance ethanol, polyhydroxylated alcohols, complexing agents, preserving agents, humectants, sweeteners, flavouring agents, colouring agents and the like.

Typical examples of these ingredients are:
polyhydroxylated alcohols: glycerol, propylene glycol and polyethylene glycol;
complexing agents: sodium edetate;
preserving agents: methyl p-hydroxybenzoate and propyl p-hydroxybenzoate, sodium benzoate;
humectants: glyceryl polyethylene glycol ricinoleate;
sweeteners: sodium saccharinate, sorbitol, acesulfame and xylitol;
gelling agents: block copolymers of polyethylene glycol and polypropylene glycol such as, for example, Poloxamer™ 407;
flavouring agents: mint flavouring agent, natural tutti frutti flavouring agent and grenadine flavouring agent;
colouring agents: quinoline yellow E 104 and patent blue E 131.

Typical examples of oropharyngeal cavity disorders which benefit from treatment with the composition of the present invention are: gingivitis, glossitis, stomatitis, aphthae, paradentosis, paradentitis, laryngitis, pharyngitis and mucositis caused by radiotherapy and chemotherapy. In addition, the composition of the invention is useful in the treatment of after-effects of dental and/or general surgery.

Preferred dosage forms of the composition of the present invention are mouthwashes and oral sprays.

These dosage forms can be readily prepared according to techniques known to pharmaceutical chemists, and include stages such as mixing, dissolution, sterilization and the like.

The following examples serve to illustrate the invention without, however, limiting it.

### EXAMPLE 1

### Mouthwash A

100 g of Mouthwash A contains:

| | | |
|---|---|---|
| salt of diclofenac with tromethamine* | 0.104 | g |
| xylitol | 10.000 | g |
| Poloxamer ™ 407 | 0.500 | g |
| sodium benzoate | 0.500 | g |
| natural mint flavouring agent | 0.500 | ml |
| aqueous solution of E 131 (1 mg/ml) | 0.200 | ml |
| pH 7.8 phosphate buffer** qs | 100 | g |
| pH | 7.6 | |

*equal to 0.074 g of acidic diclofenac
**one litre of solution in purified water contains: anhydrous dibasic sodium phosphate (5.803 g), anhydrous monobasic potassium phosphate (3.522 g) and 1N sodium hydroxide (18.70 ml).

### EXAMPLE 2

### Mouthwash B

100 g of Mouthwash B have the same composition as Mouthwash A except that:

it also contains natural tutti frutti flavouring agent (0.04 ml) and natural grenadine flavouring agent (0.02 ml), and
in place of 0.2 ml of aqueous solution of E 131 (1 mg/ml), it contains 0.25 ml of aqueous solution of E 124 (10 mg/ml).

### COMPARATIVE EXAMPLE 1

### Mouthwash C

A mouthwash was prepared having the same composition as Mouthwash A, except that it contained purified water in place of the pH 7.8 phosphate buffer.

### COMPARATIVE EXAMPLE 2

### Mouthwash D

A mouthwash was prepared having the same composition as Mouthwash B. except that it contained purified water in place of the pH 7.8 phosphate buffer.

### Stability

Mouthwashes A and B were found to be stable.

In contrast, Mouthwashes C and D released over time, especially under cold conditions, a precipitate of diclofenac.

This behaviour was entirely unexpected as regards the mouthwashes containing an amount of salt of diclofenac with tromethamine that is less than the solubility limit reported by Fini et al. (cited above).

1. Composition for the topical treatment of oropharyngeal cavity disorders, characterized in that it comprises an aqueous solution of the salt of diclofenac with tromethamine, in which the amount of the said salt is of from 0.1% to 0.2% (w/w) and the pH is adjusted between 7 and 8.

2. Composition according to claim 1, characterized in that it contains 0.10% (w/w) of the salt of diclofenac with tromethamine.

3. Composition according to claim 1 or 2, characterized in that it further comprises a sweetener selected from the group comprising sodium saccharinate, sorbitol, acesulfame and xylitol.

4. Composition according to any one of the preceding claims 1 to 3, characterized in that it further comprises a preserving agent selected from the group comprising sodium benzoate, methyl p-hydroxybenzoate and propyl p-hydroxybenzoate.

5. Composition according to any one of the preceding claims 1 to 4, characterized in that it further comprises a gelling agent consisting of a block copolymer of polyethylene glycol and polypropylene glycol.

6. Composition according to any one of the preceding claims 1 to 5, characterized in that it further comprises a pharmaceutically acceptable flavouring agent.

7. Composition according to any one of the preceding claims 1 to 6, characterized in that it further comprises a pharmaceutically acceptable colouring agent.

8. Composition according to any one of the preceding claims 1 to 7, characterized in that it is used in the treatment of gingivitis, glossitis, stomatitis, aphthae, paradentosis, paradentitis, laryngitis, pharyngitis, mucositis of the oral cavity caused by radiotherapy and chemotherapy, and of after-effects of dental and/or general surgery.
